(19) United States
(12) Patent Application Publication
(10) Pub. No.: US 2016/0002694 A1
(43) Pub. Date: Jan. 7, 2016

(54) CULTURE MEDIUM FOR GROWTH OF RECOMBINANT PROTEINS

(71) Applicant: CENTRE FOR CELLULAR AND MOLECULAR PLATFORMS, Bangalore, Karnataka (IN)

(72) Inventors: Muniasamy NEERATHILINGAM, Bangalore (IN); Narendrakumar SEKAR, Bangalore (IN); Soumya Karyidan VEETIL, Bangalore (IN); Sumukh MYSORE, Bangalore (IN); Thrinath MULLAPUDI, Bangalore (IN); Bidisha Ghosh DUTTA, Bangalore (IN); Falguni PATEL, Bangalore (IN); Rohini SHRIVASTAVA, Bangalore (IN); Deepa Kalari VEETIL, Bangalore (IN); Sneha Ganapayya BAIRY, Bangalore (IN)

(73) Assignee: CENTRE FOR CELLULAR AND MOLECULAR PLATORMS, Bangalore, Karnataka (IN)

(21) Appl. No.: 14/767,799

(22) PCT Filed: Feb. 13, 2014
(86) PCT No.: PCT/IB2014/058962

§ 371 Date: Aug. 13, 2015

(30) Foreign Application Priority Data
Feb. 13, 2013 (IN) 638/CHE/2013

(51) Int. Cl. C12P 21/00; C12N 1/16; C12N 1/20

(52) U.S. Cl. CPC C12P 21/00; C12N 1/20; C12N 1/16

(57) ABSTRACT

The present disclosure relates to a culture medium comprising coconut water and nitrogen source, optionally along with magnesium salt and excipient or a combination thereof. The present disclosure further relates to a method of preparing the culture medium for prokaryotic and eukaryotic organisms, wherein the culture medium comprises coconut water normalized by supplementation with nitrogen source which overcomes inconsistency in growth pattern and results in substantial improvement in growth and biomass of the organisms. The present disclosure further relates to successful expression of recombinant proteins by prokaryotes and eukaryotes when cultured in the said culture medium.

### TECHNICAL FIELD

The present disclosure relates to a culture medium comprising coconut water and nitrogen source, optionally along with magnesium salt and excipient or a combination thereof, a method of preparing the said culture medium and a kit thereof. Further, the disclosure relates to a method of enhancing growth or biomass concentration of prokaryotic cell or eukaryotic cell or enhancing recombinant protein expression therein by culturing the prokaryotic cell or eukaryotic cell in the said culture medium.

### BACKGROUND AND PRIOR ART OF THE DISCLOSURE

Coconut (Cocos nucifera L.) is an important fruit tree in the tropical regions. India is the largest producer of coconut with a production of 14925 million nuts annually. About 90% of the production comes from southern states, out of which the contribution of Kerala is 45% followed by Tamil Nadu 28% and Karnataka with 11%. Coconut water is the liquid endosperm present in the cavity of the fruit. During early development, coconut water remains as a suspension and as growth continues it matures into their cellular phase and deposits into the rind of the coconut meat.

Coconut water or tender coconut water constitutes of 95.5% water, 4% sugars, 0.1% fat, 0.02% calcium, 0.01% phosphorous, 0.5% iron, considerable amounts of amino acids, mineral salts, vitamin C and B complex vitamins and growth hormones (Renata Vigliar et al., 2006). Coconut water or tender coconut water contains numerous essential minerals like magnesium, potassium, boron, copper, iodine, iron, molybdenum and zinc and the vitamins like biotin, nicotinic acid, pantothenic acid and riboflavin. In addition, shikimic acids and quinic acids have been detected which play a significant role in the nutrition of developing coconut.

Traditionally coconut water or tender coconut water has been used as beverage. There are several therapeutic applications of coconut such as glucose-electrolyte oral re-hydration solution for treatment of diarrhea, intravenous hydration fluid and as probiotic drink (beneficial microflora in the gastrointestinal tract). Researchers have widely shown that coconut water due to its rich nutrient content is an excellent supplement in growth media for prokaryotic and eukaryotic cells.

General purpose culture media contain digested animal proteins as the main source of organic carbon and nitrogen. Culture media are usually supplied with peptone, meat or yeast and beef extract that provide essential ions, minerals and vitamins. These expensive nutrients amount to 30% of the total production cost.

In the prior art, coconut water has been used as media supplement for plant tissue culture media. European Patent Application EP0229174 disclosed the use of coconut water (10 percent v/v) in basal medium of Murashige and Skoog for transgenic plant cell culture.

WIPO Patent Application WO2010/037208A1 used XB-4 protoplast medium supplemented with coconut water and other factors for production of pharmaceutically active molecules in plant cell culture.

US patent Publication number 2010/0126945 disclosed the preparation of bacterial cellulose films with matured coconut water. The water was first boiled and then Sucrose, (65 g L^{′1}) was dissolved and 25 g L^{′1} acetic acid was added.

WIPO Patent Application WO/2002/040043 discloses a method for extracting active ingredients from the liquid endosperm of fresh green coconuts.

Oloke and Glick (2006) showed that endogenous proteins can be expressed in E. coli using coconut water as media. Seesuriyachan et. al (2011) used coconut water supplemented with 20 g/l crystalline sucrose and a reduced quantity of the three expensive supplements (5 g/l of peptone, 2.5 g/l of yeast extract, and 2.5 g/l of beef extract) to produce exopolysaccharide (EPS) using Lactobacillus confusus TISTR 1498. Although the above studies focused on growth of microbes and endogenous protein production in coconut water, its application in recombinant protein production has not been demonstrated before.

From the prior art we know that coconut water is used as a fixed percentage of the total media along with other animal protein for promoting growth in medium. The present disclosure discloses a process for modification of coconut water or tender coconut water in order to serve as complete medium for prokaryotes such as bacteria and eukaryotes such as yeast. Instead of employing several nutritional components, coconut water can be used as sole medium. The advantages of this medium are wide availability, cost efficiency and green alternative to animal based protein products.

According to Hiroshi Nikaido (Nov. 9, 2009), while LB media may appear as a suitable media for many bacteria of research interest, it is an inappropriate choice for physiological studies wherein reproducibility is required. Since only bacterial cultures in balanced growth (achieved by sufficient time in exponential growth) have a reproducible average cell size and chemical composition, none of the components of liquid media should become exhausted during growth of the culture. Growth of E. coli usually stops, even in the presence of the large total concentration of organic nutrients in LB broth, when the OD₆₀₀ reaches around 2, corresponding to about 0.6 mg of E. coli (dry weight) per ml. The reason is not difficult to find: LB medium provides only a scant amount of carbohydrates, and surprisingly small amounts of other utilizable carbon sources. LB media sold as premixed capsules by certain companies contain bile salts as contaminate of the “beef” product present in the medium. The contamination is sufficient to prevent the growth of mutants that lack the multidrug efflux pump (AcrAB-TolC) of E. coli, one function of which is to protect the cells from bile salts in their environment. This unpleasant fact speaks of the differences between batches of premixed medium. Unless made from scratch, LB broth can vary from batch to batch, thus cannot be considered reproducible. The bottom line is that, from a multitude of perspectives, the use of LB medium is to be discouraged, especially for use in any studies in which the physiological state and metabolic functions of the cell matter.

In addition, in the prior art there is no discussion to overcome batch-to-batch variation in the nutrient content of the coconut. Unagul et al., 2007 have reported 10% variation in content of chemical components of coconut water. There are over 80 different varieties of coconut palm present world-wide. Jackson et al. showed that coconut water of different coconut varieties contains different concentration of compounds, and that the chemical contents also varied during the different stages of maturity. Soil and environmental conditions also affect the chemical profile of coconut water. A study which was done in Brazil demonstrated that the properties of coconut water were affected by varying nitrogen and potassium application (Young et. al. 2009). Thus the quality of coconut varies from sample to sample. This study does not report nitrogen as supplement in CW for growth of microbes. Instead, the study involves profiling of the composition of coconut water in which potassium, nitrogen and other compounds were found to vary.

For large-scale experiment coconuts collected from varied location shall be present at different state of maturity resulting in disparity in growth pattern. Further, in recent years, the therapeutic application of recombinant proteins has been increased immensely. Many industries are producing large quantities of recombinant proteins and the need for expression media devoid of any toxin has increased subsequently.

The present disclosure is targeted at addressing such limitations existing in the prior known techniques in the same field of technology.

In the current disclosure, as nitrogen is the limiting factor, the coconut water (CW) is normalised to obtain uniform growth. As a result of normalisation there is marked increase in biomass of prokaryotes such as bacteria and Eukaryotes such as yeast. This standardized/normalized medium is further used for expression of recombinant protein.

### STATEMENT OF THE DISCLOSURE

Accordingly, the present disclosure relates to a culture medium comprising coconut water and nitrogen source, optionally along with magnesium salt and excipient or a combination thereof; a method of preparing culture medium, said method comprising act of combining coconut water with nitrogen source, optionally along with magnesium salt and excipient or a combination thereof, for preparing said culture medium; a method of enhancing growth or biomass concentration of prokaryotic cell or eukaryotic cell or enhancing protein expression therein or any combination thereof, said method comprising act of culturing the prokaryotic cell or eukaryotic cell in a culture medium comprising coconut water and nitrogen source, optionally along with magnesium salt and excipient or a combination thereof; a culture medium for use in enhancing growth or biomass concentration of prokaryotic cell or eukaryotic cell or enhancing recombinant protein expression therein or any combination thereof, wherein said culture medium comprises coconut water and nitrogen source, optionally along with magnesium salt and excipient or a combination thereof; and a kit comprising culture media having coconut water and nitrogen source optionally along with magnesium salt and excipient or a combination thereof, along with an instruction manual.

### BRIEF DESCRIPTION OF THE ACCOMPANYING FIGURES

In order that the disclosure may be readily understood and put into practical effect, reference will now be made to exemplary embodiments as illustrated with reference to the accompanying figures. The figures together with a detailed description below, are incorporated in and form part of the specification, and serve to further illustrate the embodiments and explain various principles and advantages, in accordance with the present disclosure wherein:

FIG. 1 shows Growth curve of (A) E. coli and (B) P. pastoris in CW supplemented (CW-S) with ammonium sulphate, (C) Growth of E. coli C41 on LB agar plate (left) and CW agar plate (right). (D) (NH₄)₂SO₄ supplementation in five different CW samples (CW-S) and comparison with its corresponding CW.

FIG. 2 (A) shows Growth curve of E. coli in CW-S supplemented with (NH₄)₂SO₄ at different concentration (25, 37 & 50 mM) and 2 mM MgSO₄ in combination with 25 mM (NH₄)₂SO₄ and 0.36% amino acid mix and FIG. 2(B) Growth curve of Pichia in sole CW, CW supplemented with (NH₄)₂SO₄, KH₂PO₄ and amino acid mix.

FIG. 3 shows Recombinant protein expression in E. coli grown in CW media. Protein expression was analysed in 12% SDS-PAGE. (A) MBP-TEV protease expression at 69 KDa harvested after 5 hours (B) MBP expressed at 42 KDa harvested after 5 hours (C) mEos2 expressed at 27 KDa harvested after overnight induction. All samples normalized based on their OD₆₀₀. Lane M: Marker, Lane U: Uninduced, Lane I: Induced. MBP & MBP-TEV constructs were induced with 0.4 mM IPTG while mEos2 with 0.1 mM IPTG.

FIG. 4 shows Recombinant protein expression in E. coli grown in CW-S Protein expression in CW supplemented with (NH₄)₂SO₄ and its comparison to the expression in LB media. (A) MBP (42 KDa) and mEos2 (27 KDa) expression in LB media (B) MBP and mEos2 expression in CW supplemented with (NH₄)₂SO₄. Lane M: Marker, Lane U: Uninduced, Lane I: Induced.

### DETAILED DESCRIPTION OF THE DISCLOSURE

The present disclosure relates to a culture medium comprising coconut water and nitrogen source, optionally along with magnesium salt and excipient or a combination thereof.

In an embodiment of the present disclosure, the coconut water is obtained from coconut fruit by pooling from multiple coconuts or from single coconut individually.

In another embodiment of the present disclosure, the nitrogen source is selected from a group comprising ammonium sulphate, ammonium acetate, ammonium carbonate, ammonium chloride and amino acid mix or any combination thereof.

In yet another embodiment of the present disclosure, the nitrogen source is ammonium sulphate at concentration ranging from about 10 mM to about 100 mM, preferably at about 25 mM.

In still another embodiment of the present disclosure, the magnesium salt is selected from a group comprising magnesium chloride, magnesium carbonate and magnesium sulphate; and wherein the magnesium salt is at concentration ranging from about 1 mM to about 10 mM, preferably about 2 mM.

In still another embodiment of the present disclosure, the excipient is selected from a group comprising inducer, activator and antibiotic or any combination thereof.

In still another embodiment of the present disclosure, the excipient is IPTG and the antibiotic is ampicillin.

In still another embodiment of the present disclosure, the culture medium has pH ranging from about 4 to about 8.

The present disclosure also relates to a method of preparing culture medium, said method comprising act of combining coconut water with nitrogen source, optionally along with magnesium salt and excipient or a combination thereof, for preparing said culture medium.

In an embodiment of the present disclosure, the coconut water is extracted from a single coconut fruit or multiple coconut fruits; and upon said extraction, solid particles are optionally removed from the coconut water followed by aseptic sterilization or by filter sterilizing said water before combining with the nitrogen source for preparing the culture medium.

In another embodiment of the present disclosure, the coconut water extracted from multiple coconut fruits is pooled before optional removal of solid particles and filter sterilization.

In yet another embodiment of the present disclosure, the removal of the solid particles from the coconut water is carried out by centrifugation at about 3000 rpm to about 10000 rpm for time duration ranging from about 15 min to about 30 min.

In still another embodiment of the present disclosure, the filter sterilization is carried out using a membrane filter having pore size ranging from about 0.01 μm to about 0.30 μm, preferably about 0.2 μm.

The present disclosure also relates to a method of enhancing growth or biomass concentration of prokaryotic cell or eukaryotic cell or enhancing recombinant protein expression therein or any combination thereof, said method comprising act of culturing the prokaryotic cell or eukaryotic cell in a culture medium comprising coconut water and nitrogen source, optionally along with magnesium salt and excipient or a combination thereof.

In an embodiment of the present disclosure, the prokaryotic cell is Escherichia coli and the eukaryotic cell is Pichia pastoris.

In another embodiment of the present disclosure, the culture medium has pH ranging from about 4 to about 8.

In yet another embodiment of the present disclosure, the culture medium enhances the growth or the biomass concentration of the Escherichia coli by about 2 to about 10 folds and enhances the growth or the biomass concentration of the Pichia pastoris by about 1.5 to about 3 folds in comparison to respective growth or biomass concentration when grown in conventional media.

In still another embodiment of the present disclosure, the culture medium facilitates the protein expression of recombinant protein selected from a group comprising MBP, MBP-TEV and mEos2 or any combination thereof in Escherichia coli or Pichia pastoris.

In still another embodiment of the present disclosure, the prokaryotic cell or the eukaryotic cell is grown in the culture medium by a method selected from a group comprising batch-cultivation, perfusion cultivation and chemostat cultivation.

The present disclosure also relates to a culture medium for use in enhancing growth or biomass concentration of prokaryotic cell or eukaryotic cell or enhancing protein expression therein or any combination thereof, wherein said culture medium comprises coconut water and nitrogen source, optionally along with magnesium salt and excipient or a combination thereof.

The present disclosure also relates to a kit comprising culture media having coconut water and nitrogen source optionally along with magnesium salt and excipient or a combination thereof; along with an instruction manual.

In an embodiment of the present disclosure, the nitrogen source is selected from a group comprising ammonium sulphate, ammonium acetate, ammonium carbonate, ammonium chloride and amino acid mix or any combination thereof; wherein the magnesium salt is selected from a group comprising magnesium chloride, magnesium carbonate and magnesium sulphate; and wherein the excipient is selected from a group comprising inducer, activator and antibiotic or any combination thereof.

In another embodiment of the present disclosure, the nitrogen source is at concentration ranging from about 10 mM to about 100 mM, the magnesium salt is at concentration ranging from about 1 mM to about 10 mM, and excipient is at appropriate concentration.

The principal object of the instant disclosure is supplementation of Coconut Water (CW) which is obtained from various parts of South India, with inorganic nitrogen sources like ammonium sulphate to overcome inconsistency in growth pattern in different CW Another object, of the instant disclosure is to increase biomass production of prokaryotes such as bacteria when Ammonium salt is added to the medium as compared to employing sole or un-supplemented Coconut Water. It is still further object of the present disclosure to increase biomass production of Eukaryotes such as Pichia using the modified medium or supplemented CW. A further object of the instant disclosure is that the supplemented media is used for expression of three Recombinant proteins in E. coli demonstrated by expression of Maltose Binding Protein (MBP, 42 KDa), a fusion of MBP-Tobacco Etch Virus protease (69 KDa), and mEos2 (27 KDa), which are induced using appropriate concentration of IPTG. Yet another object of the instant disclosure is to prepare coconut water agar plates for growing the transformants of E. coli.

The present disclosure relates to improvements in culture media, in which a substitution for animal free culture medium is provided by employing plant based natural media, for high production of biomass and over-expression of recombinant proteins.

Another embodiment of the present disclosure relates to a method of formulating a culture medium for prokaryotes such as E. coli and eukaryotes such as Pichia pastoris as model organisms for enhanced biomass production/growth and increased expression of recombinant proteins in said organisms, using supplemented coconut water.

Yet another embodiment of the present disclosure relates to use of animal product free media containing simple salt supplements for growth of cells similar to cells growing in conventional animal protein containing media and wherein such animal free media facilitates enhanced expression of physiologically active recombinant proteins such as Maltose binding protein (MBP), Maltose binding protein-tobacco etch virus protease (MBP-TEV) protease fusion and a photo switchable florescent protein (mEos2) in E. coli.

In still another embodiment of the present disclosure, the medium is prepared from water obtained from coconuts present from young to mature stage preferably without the pulp.

Still another embodiment of the present disclosure relates to addition of nitrogen salts to the animal product free culture medium to normalize the variation in growth pattern and increase the biomass or cell growth of the prokaryotes and eukaryotes when cultured in said medium.

Still another embodiment of the present disclosure relates to addition of ammonium sulphate—(NH₄)₂SO₄ to animal product free culture medium in the concentration range of about 10 mM to about 100 mM, preferably less than about 40 mM in order to normalize the different rate of growth and increase biomass/cell growth productivity.

Still another embodiment of the present disclosure relates to optional supplementation of the animal free culture medium with magnesium sulphate—MgSO₄ in the concentration range of about 1 mM to about 10 mM, preferably less than about 5 mM in order to improve biomass productivity.

Still another embodiment of the present disclosure relates to 2 fold increase in cell growth or biomass productivity in case of E. coli and 1.5 to 2 fold increase in case of P. pastoris when supplemented coconut water medium is used in the animal-free culture medium thus enhancing the productivity of recombinant proteins.

Still another embodiment of the present disclosure relates to use of coconut water containing simple salt supplements for favorable growth of cells, in the range of pH about 4 to about 8.

Still another embodiment of the present disclosure relates to use of coconut water containing simple salt supplements for favorable growth of cells, in the temperature range of about 25° C. to about 37° C.

In still another embodiment of the present disclosure, the culture medium is prepared by dissolving aforementioned ingredients in coconut water or tender coconut water in their respective concentrations and filtering obtained solution/mixture on membrane filter with aid of vacuum pressure at about 550 to about 600 mmHg to obtain sterilized culture medium.

The present disclosure also has many advantages over current methods such as recombinant protein expression in E. coli under the control of lac promoter. Further, the present disclosure describes a simple methodology for obtaining recombinant proteins, where the absence of lactose favors no leaky expression in case of lac-operon based systems. Expression levels of all proteins are comparable to that of LB media thus, implying that supplemented-coconut water or supplemented-tender coconut water (with nitrogen sources) can act as a potential media for recombinant protein expression as well. In general, coconut water (CW) is natural, naturally sterile, the preparation and sterilization is fairly convenient and easy in comparison to conventionally used media, it is economical or in-expensive and abundantly available throughout the year (especially in tropical and coastal areas), thereby contributing to its advantages and feasibility as a potential microbial growth medium.

A more complete understanding can be obtained by reference to the following specific examples, which are provided for purposes of illustration only and are not intended to limit the scope of the disclosure.

### EXAMPLES

### Example 1

### Extraction and Sterilization of Coconut Water (CW)

Coconut is collected and part of its mesocarp and endocarp are removed to expose the surface of endosperm. Then, coconut water (CW) present therein is collected in sterile container and centrifuged at about 4000 rpm for about 20 minutes to remove any solid particles, followed by filter sterilization with about 0.22 μm filter. On an average, 300 to 500 ml of CW per coconut can be obtained from one coconut fruit. pH of the CW is noted as 4.6-4.8.

### Example 2

### Normalization of CW for Consistent Growth and Increase in Biomass

The varying nitrogen content in different CW samples is confirmed through methods such as Kjeldahl method for total nitrogen and mass spectroscopic analysis of individual amino acid (Table 1):

Table 1 shows the chemical profile of CW used in the present study

| | CW | 1 CW | 2 CW | 3 CW | 4 CW | 5 CW | 6 SD* |
|---|---|---|---|---|---|---|---|
| Compounds | (μg/ml) | (μg/ml) | (μg/ml) | (μg/ml) | (μg/ml) | (μg/ml) | (μg/ml) |
| Hydroxyproline | 1.80 | 2.41 | 2.52 | 2.18 | 2.31 | 2.39 | ±0.23 |
| Histidine | 2.03 | 2.57 | 2.94 | 1.83 | 3.61 | 3.04 | ±0.60 |
| Asparsgine | 176.88 | 98.07 | 68.18 | 97.60 | 223.48 | 196.21 | ±57.92 |
| Taurine | 0.07 | 0.07 | 0.14 | 0.09 | 0.09 | 0.09 | ±0.02 |
| Serine | 8.27 | 8.03 | 8.40 | 7.01 | 11.27 | 10.36 | ±1.45 |
| Glutamine | 6.44 | 3.32 | 2.83 | 5.81 | 5.11 | 12.49 | ±3.17 |
| Arginine | 12.49 | 19.09 | 17.70 | 21.49 | 26.91 | 22.36 | ±4.43 |
| Homoserine | 1.30 | 1.39 | 1.06 | 1.16 | 1.31 | 1.33 | ±0.11 |
| Glycine | 2.90 | 2.78 | 2.80 | 2.39 | 3.00 | 3.98 | ±0.48 |
| Aspartic acid | 0.52 | 0.25 | 0.50 | 0.31 | 0.94 | 0.71 | ±0.23 |
| Citrulline | 1.02 | 1.37 | 1.20 | 1.10 | 1.29 | 1.23 | ±0.11 |
| Glutamic acid | 3.43 | 1.50 | 4.93 | 1.88 | 6.22 | 5.00 | ±1.71 |
| Threonine | 10.53 | 12.25 | 11.07 | 5.67 | 10.63 | 10.79 | ±2.08 |
| Alanine | 193.56 | 171.85 | 252.66 | 166.57 | 248.37 | 237.76 | ±35.71 |
| Gaba | 15.78 | 14.51 | 33.24 | 22.70 | 30.18 | 28.18 | ±7.07 |
| Proline | 20.78 | 56.54 | 50.26 | 17.23 | 31.98 | 34.73 | ±14.28 |
| Lysine | 0.92 | 1.27 | 1.96 | 1.50 | 2.14 | 1.71 | ±0.41 |
| Tyrosine | 6.69 | 3.48 | 4.27 | 4.00 | 10.32 | 9.02 | ±2.61 |
| Methonine | 0.03 | 0.04 | 0.06 | 0.16 | 0.09 | 0.11 | ±0.04 |
| Valine | 54.69 | 52.16 | 48.32 | 52.79 | 64.44 | 62.40 | ±5.74 |
| Isoleucine | 15.48 | 15.72 | 14.53 | 14.89 | 18.01 | 17.74 | ±1.34 |
| Lucine | 29.35 | 24.09 | 20.21 | 25.30 | 38.48 | 34.71 | ±6.28 |
| Phenylalanine | 7.28 | 5.03 | 5.07 | 4.80 | 8.66 | 7.81 | ±1.53 |
| Total amino | 576.64 | 498.83 | 704.50 | 457.25 | 759.32 | 557.02 | ±107.19 |
| acids | | | | | | | |
| metabolites¹ | | | | | | | |
| Total | 3.9 g/ | 3.82 g/ | 2.82 g/ | 2.80 g/ | 2.32 g/ | 2.63 g/ | ±0.611 g/ |
| carbohydrate² | 100 ml | 100 ml | 100 ml | 100 ml | 100 ml | 100 ml | 100 ml |
| Total | 33.2 mg/ | 17.542 mg/ | 33.60 mg/ | 15.0 mg/ | 57.2 mg/ | 28.630 mg/ | ±13.78 mg/ |
| Nitrogen³ | 100 ml | 100 ml | 100 ml | 100 ml | 100 ml | 100 ml | 100 ml |

Amino acids and metabolites listed in the table are estimated using mass spectroscopy.
¹Sum of amino acid and other metabolites concentration is estimated by mass spectroscopy.
²Total carbohydrate was estimated by phenol sulphuric acid method.
³Total nitrogen is estimated by Kjeldahl method.
*SD—standard deviation.

Nitrogen source in the form of ammonium based salts such as ammonium sulphate, ammonium acetate, ammonium carbonate or ammonium chloride is added to CW to obtain consistent biomass in different coconut water samples.

The normalization of CW from individual samples and pooled samples having nitrogen source deficiency is observed by supplementing the same with ammonium salts. The inconsistency of growth in pooled CW as well as in CW of individual coconuts can be normalized by the same method.

The supplementation of one or more CW with ammonium salts is performed by any one of the below two approaches:
1. CW samples collected from different locations are pooled or mixed together followed by the addition of ammonium based salts.
2. CW samples are collected from different locations, to which ammonium based salt is added individually after which the samples are mixed together to obtain the CW broth.

### Example 3

### Preparation of CW Agar Plates

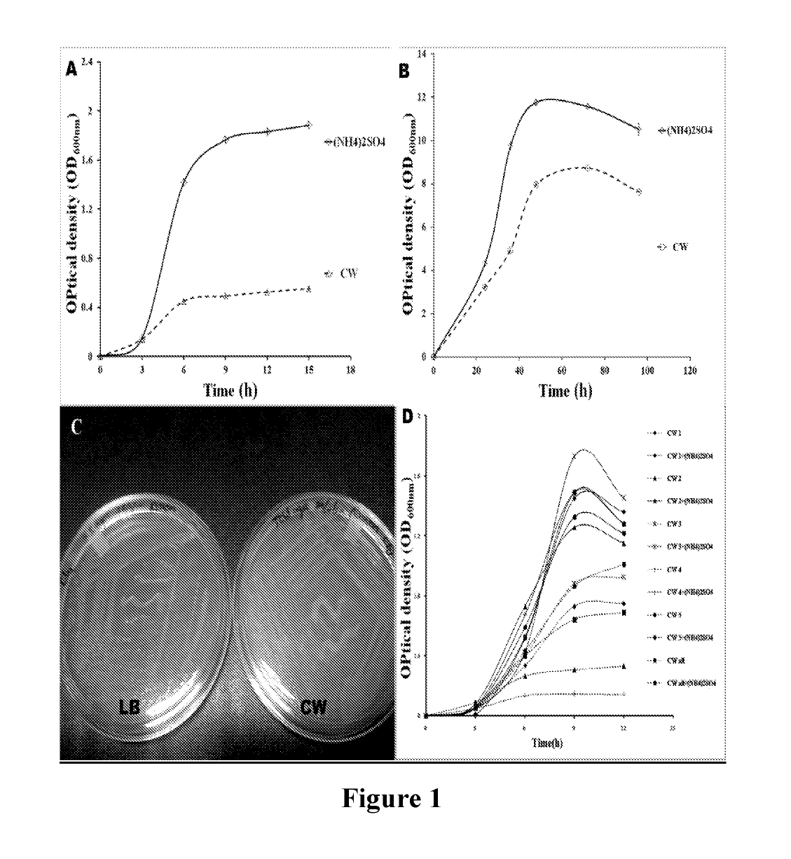

Equal volume of autoclaved 4% agar is mixed with filter sterilized sole coconut water and micro-waved for 30-60 seconds to ensure proper mixing. 4% agar is also mixed with filter sterilized coconut water which is supplemented with nitrogen salt and micro waved for 30-60 seconds to ensure proper mixing. This is then poured on petri plates and allowed to solidify. Loop full of E. coli culture is streaked on the CW agar and incubated at 37° C. overnight (FIG. 1C).

### Example 4

### Culturing of E. coli in Sole CW and in Normalized/Supplemented CW

The cumulative effect of supplementation of CW with different concentration of the ammonium salt—(NH₄)₂SO₄, (NH₄)₂SO₄ with MgSO₄ and amino acid with MgSO₄, is noted separately as described below.

A loopfull of E. coli is inoculated from glycerol stock/plate into 10 ml of sole and supplemented CW. The supplemented CW is divided into 3 sets as follows: The samples of the first set are supplemented with different concentration of ammonium salt—(NH₄)₂SO₄ (i.e. 25 mM, 37 mM and 50 mM), the second set is supplemented with 25 mM (NH₄)₂SO₄ and MgSO₄ and the third set is supplemented with amino acid mix and MgSO₄. All the sets of broths are further incubated at 37° C. overnight with 200 rpm in a shaker incubator. For biomass estimation one percent of seed i.e. the primary culture (overnight culture) is inoculated in 10 ml of CW and incubated at 37° C. in a shaker incubator with 200 rpm. OD₆₀₀ nm is measured at every 3 hours interval (FIG. 1A).

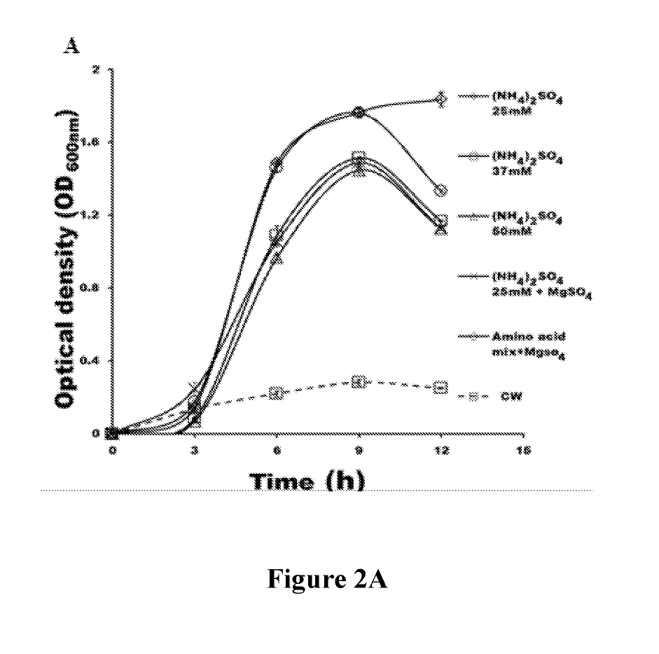

The biomass of E. coli is decreased in (NH₄)₂SO₄+MgSO₄ whereas, amino acids+MgSO₄ supplementation is equivalent to that of the 25 mM (NH₄)₂SO₄. Further to check whether increase in concentration of ammonium sulphate improves the biomass of E. coli, CW is supplemented with 25 mM, 37 mM and 50 mM of (NH₄)₂SO₄ and compared with CW without supplementation. A six fold increase is observed with 25 mM (NH₄)₂SO₄ supplementation while only 4.5 fold increase is noted in 37 mM as well as in 50 mM (NH₄)₂SO₄ supplemented CW (FIG. 2). Above results suggest that the supplementation of 25 mM (NH₄)₂SO₄ is sufficient to achieve the improved and consistent growth.

Further, to validate the ammonium sulphate supplementation for normalizing batch to batch variation of CW, five samples from different locations are tested.

A loop-full of E. coli culture is inoculated into 10 ml of sole CW broth from 5 different sources (1^{st} set), in 10 ml of CW broth from 5 different sources supplemented by the addition of nitrogen source individually to each sample (2^{nd} set), in 10 ml of sole CW broth pooled from 5 different sources (3rd set) and in 10 ml of CW broth pooled from 5 different sources which is normalized by the addition of nitrogen source (4^{th} set). These are incubated at 37° C. overnight with 200 rpm in a shaker incubator. For biomass estimation one percent of seed i.e. the primary culture (overnight culture) is inoculated in 10 ml of CW and incubated at 37° C. in a shaker incubator with 200 rpm. OD_{600 n}m is measured at every 3 hours interval (FIG. 1D).

Table 2 shows the fold increase of growth of E. coli at OD₆₀₀ nm maximum which was noted as 9^{th} hour from FIG. 1D.

| | CW | CW-S | Fold Increase |
|---|---|---|---|
| CW1 | 0.73 | 1.45 | 1.97 |
| CW2 | 0.308 | 1.256 | 4.08 |
| CW3 | 0.88 | 1.728 | 1.93 |
| CW4 | 0.146 | 1.488 | 10.19 |
| CW5 | 0.764 | 1.46 | 1.91 |
| CW all | 0.652 | 1.488 | 2.32 |

A 2-10 fold increase in biomass of E. coli in 25 mM (NH₄)₂SO₄ supplemented CW is observed as compared to CW without supplementation (FIG. 1D). Also, when these five samples are pooled together and OD_{600 nm} 0.6 is noted while, upon addition of 25 mM (NH₄)₂SO₄ it increases to 1.2. This signifies that the supplementation of CW with 25 mM (NH₄)₂SO₄ can be used for large scale applications as well, since several coconut fruits would be required to obtain sufficient volume of CW.

Supplementation of CW with other ammonium based salts such as ammonium acetate, ammonium carbonate or ammonium chloride at concentration ranging from about 10 mM to about 100 mM also causes 2-10 fold increase in the biomass of E. coli.

Biomass of Pichia in CW broth is estimated by inoculating 10 μl of glycerol stock which is prepared by adding 15% glycerol into overnight grown culture and stored at −80° C., into 10 ml of sole CW broth and supplemented CW broth and incubating at 30° C. by shaking at 200 rpm. OD₆₀₀ nm is measured at every 12 hours interval. FIG. 1B shows the growth curve of Pichia pastoris cultured in CW and supplemented CW.

### Example 5

### Expression of Recombinant Proteins in CW

To demonstrate the protein expression in E. coli using CW, three different recombinant proteins are chosen. MBP (42 KDa) is a complex regulatory and transport system used in fusion to the protein of interest to increase the solubility of recombinant proteins. MBP is thus, an easily expressible target in E. coli. A longer (69 KDa) construct, which is made from the fusion of MBP and TEV protease (a highly site-specific cysteine protease commonly used for removing affinity tags from purified proteins) is taken as a second construct. The third protein of interest mEoS2 (27 KDa) is a fluorescent protein, which gives a visually detectable green color ensuring the proteins produced are in the native fold and functional.

### Strains and Plasmids:

E. coli strains such as BL21 (DE3), BL21 (DE3) pLysS are used for protein expression studies whereas, E. coli C41 (DE3) and P. pastoris GS115 are used for growth studies. All the strains are procured from Invitrogen™, USA. Three different constructs are used namely; maltose binding protein (MBP), a fusion of MBP with tobacco etch virus protease (MBP-TEV) and monomeric variant of photo switchable fluorescent Eos proteins (mEos2). pMAL-c5× for expressing MBP, pMAL-c5× harboring TEV for expressing MBP-TEV fusion and pRSET-A harbouring mEos2 are used in this study. TEV is synthesized by GeneScripts, USA and mEos2 is a kind gift from Dr. Satyajit Mayor, NCBS, Bangaluru, India.

The details on the vector constructs are as given below:

| Recombinant | Source/GenBank |
|---|---|
| protein | accession no. |
| MBP | pMALc5X vector |
| TEV | L38714.1 |
| mEos2 | FJ707374.1 |

For expression of recombinant proteins in E. coli, one percent of overnight culture is inoculated in 50 ml of sole CW (i.e. unmodified CW devoid of nitrogen salt) and 50 ml of CW supplemented with nitrogen salt. Both the cultures are then incubated at 37° C. with 200 rpm and induced with IPTG concentrations ranging from about 0.1 mM to about 1 mM at OD₆₀₀ nm ranging between 0.4-0.6 (i.e. referring to the biomass of E. coli). The cultures are further incubated at 30° C. for five hours (in case of MBP-TEV and MBP) and overnight induction (in case of mEoS2), in the shaker incubator with 200 rpm. Thereafter, the protein expression is observed in SDS-PAGE.

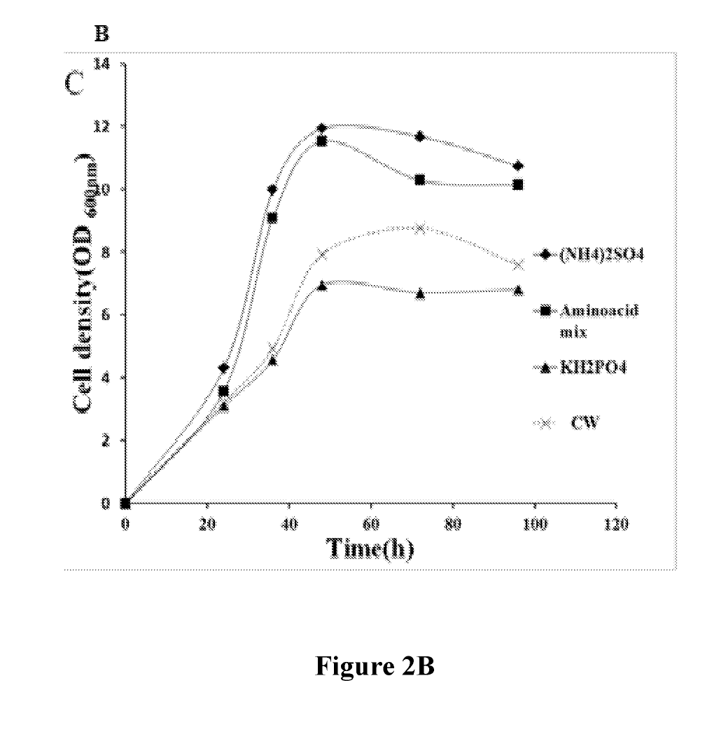

The constructs are induced using appropriate concentration of IPTG where MBP and MBP-TEV protease is controlled by tac promoter (Ptac) and mEoS2 is controlled by T7 promoter. Successful expression of the above proteins is confirmed by 12% SDS-PAGE (FIG. 3). The expression of the proteins by the induced cultures is compared with that of uninduced cultures.

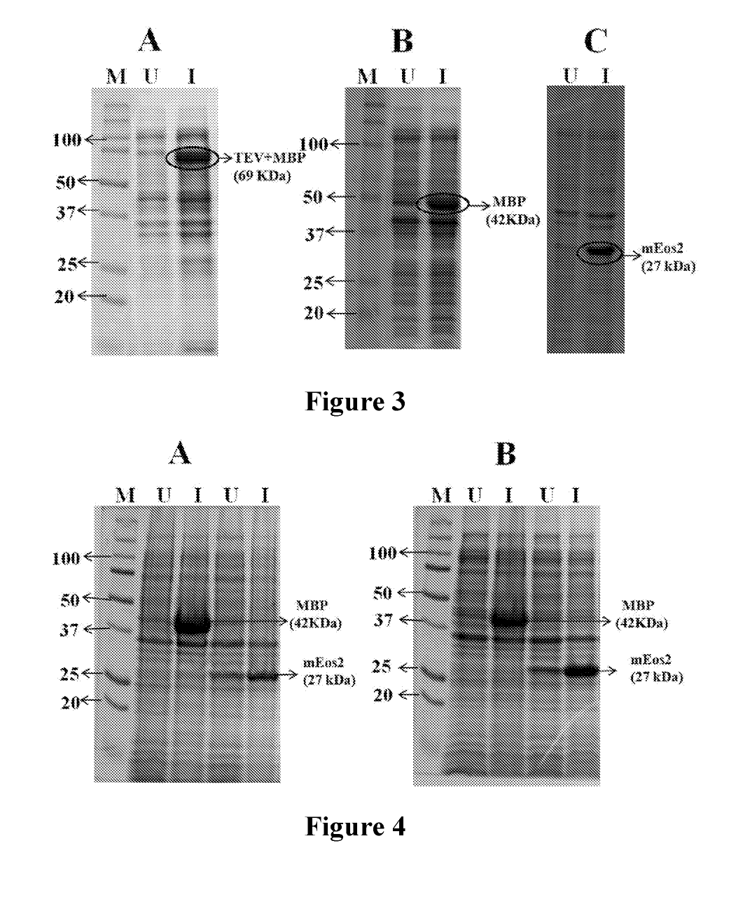

The biomass of E. coli is not consistent in different CW, which is proportionately related to protein expression. Hence, protein expression is carried out using CW-S [supplemented with 25 mM (NH₄)₂SO₄]. As compared to LB, expression level of mEoS2 is high in CW-S with less leaky expression. While for MBP, no leaky expression is observed and the expression level is almost equivalent to LB (FIG. 4).

Successful expression of MBP, MBP-TEV and mEos2 is observed in CW and CW-S which is comparable to that of expression in LB. However, difference in growth rate of E. coli and P. pastoris is observed as there is inconsistency of nitrogen source in CW, which is normalized by the supplementation of CW with 25 mM (N₄)₂SO₄. Therefore, the use of CW alone is not advisable. In order to obtain consistency of growth, 25 mM (NH₄)₂SO₄ supplementation is recommended.

The CW media which is supplemented with ammonium salt is used for the growth of competent cells wherein ¹⁵N isotope labeled ammonium salts can be used for expressing ¹⁵N labeled protein instead of normal ammonium salt for NMR studies.

1. A culture medium consisting of coconut water and nitrogen source, optionally along with magnesium salt, excipient or a combination thereof.

2. The culture medium as claimed in claim 1, wherein the coconut water is obtained from coconut fruit by pooling from multiple coconuts or from single coconut individually.

3. The culture medium as claimed in claim 1, wherein the nitrogen source is selected from a group comprising ammonium sulphate, ammonium acetate, ammonium carbonate, ammonium chloride and amino acid mix or any combination thereof.

4. The culture medium as claimed in claim 1, wherein the nitrogen source is ammonium sulphate at concentration ranging from about 10 mM to about 100 mM, preferably at about 25 mM.

5. The culture medium as claimed in claim 1, wherein the magnesium salt is selected from a group comprising magnesium chloride, magnesium carbonate and magnesium sulphate; and wherein the magnesium salt is at concentration ranging from about 1 mM to about 10 mM, preferably about 2 mM.

6. The culture medium as claimed in claim 1, wherein the excipient is selected from a group comprising inducer, activator and antibiotic or any combination thereof.

7. The culture medium as claimed in claim 1, wherein said culture medium has pH ranging from about 4 to about 8.

8. A method of preparing a culture medium as claimed in claim 1, said method comprising act of combining coconut water with nitrogen source, optionally along with magnesium salt, excipient or a combination thereof, for preparing said culture medium.

9. The method as claimed in claim 8, wherein the coconut water is extracted from a single coconut fruit or multiple coconut fruits; and upon said extraction, solid particles are optionally removed from the coconut water followed by aseptic sterilization or by filter sterilizing said water before combining with the nitrogen source for preparing the culture medium.

10. The method as claimed in claim 9, wherein the coconut water extracted from multiple coconut fruits is pooled before optional removal of solid particles and filter sterilization.

11. The method as claimed in claim 9, wherein the removal of the solid particles from the coconut water is carried out by centrifugation at about 3000 rpm to about 10000 rpm for time duration ranging from about 15 min to about 30 min.

12. The method as claimed in claim 9, wherein the filter sterilization is carried out using a membrane filter having pore size ranging from about 0.01 μm to about 0.30 μm, preferably about 0.2 μm.

13. A method of enhancing growth or biomass concentration of prokaryotic cell or eukaryotic cell or enhancing protein expression therein or any combination thereof, said method comprising act of culturing the prokaryotic cell or eukaryotic cell in a culture medium consisting of coconut water and nitrogen source, optionally along with magnesium salt, excipient or a combination thereof.

14. The method as claimed in claim 13, wherein the prokaryotic cell is Escherichia coli and the eukaryotic cell is Pichia pastoris; and wherein the culture medium enhances the growth or the biomass concentration of Escherichia coli by about 2 to about 10 folds and enhances the growth or the biomass concentration of the Pichia pastoris by about 1.5 to about 3 folds in comparison to respective growth or biomass concentration when grown in conventional media.

15. The method as claimed in claim 13, wherein the culture medium has pH ranging from about 4 to about 8.

16. (canceled)

17. The method as claimed in claim 13, wherein the culture medium facilitates the protein expression of recombinant protein selected from a group comprising MBP, MBP-TEV and mEos2 or any combination thereof in Escherichia coli or Pichia pastoris.

18. The method as claimed in claim 13, wherein the prokaryotic cell or the eukaryotic cell is grown in the culture medium by a method selected from a group comprising batch-cultivation, perfusion cultivation and chemostat cultivation.

19. A culture medium as claimed in claim 1 for use in enhancing growth or biomass concentration of prokaryotic cell or eukaryotic cell or enhancing protein expression therein or any combination thereof.

20. A kit comprising culture media and instruction manual, wherein the culture media consists of coconut water and nitrogen source optionally along with magnesium salt, excipient or a combination thereof.

21. The kit as claimed in claim 20, wherein the nitrogen source is selected from a group comprising ammonium sulphate, ammonium acetate, ammonium carbonate, ammonium chloride and amino acid mix or any combination thereof; wherein the magnesium salt is selected from a group comprising magnesium chloride, magnesium carbonate and magnesium sulphate; and wherein the excipient is selected from a group comprising inducer, activator and antibiotic or any combination thereof; and wherein the nitrogen source is at concentration ranging from about 10 mM to about 100 mM, the magnesium salt is at concentration ranging from about 1 mM to about 10 mM, and the excipient is at appropriate concentration.

22. (canceled)
